(19) United States
(12) Patent Application Publication
(10) Pub. No.: US 2025/0185665 A1
(43) Pub. Date: Jun. 12, 2025

(54) DISINFECTANT AND METHODS OF PREPARATION THEREOF

(71) Applicants: Uday Didar Singh PAUL, Dera Bassi (IN); Narinder SINGH, Rupnagar (IN)

(72) Inventors: Uday Didar Singh PAUL, Dera Bassi (IN); Narinder SINGH, Rupnagar (IN)

(21) Appl. No.: 18/846,681

(22) PCT Filed: Mar. 19, 2023
(86) PCT No.: PCT/IN2023/050266

§ 371 Date: Sep. 13, 2024

(30) Foreign Application Priority Data
Mar. 19, 2022 (IN) 202211015096

(51) Int. Cl. A01N 59/20; A01N 25/30; A01N 31/02; A01N 33/12; A01N 35/04; A01N 41/02; A01N 59/00; A01P 1/00

(52) U.S. Cl. CPC A01N 59/20; A01N 25/30; A01N 31/02; A01N 33/12; A01N 35/04; A01N 41/02; A01N 59/00; A01P 1/00

(57) ABSTRACT

The invention relates to antimicrobial and antiviral formulation and its method of preparation thereof. The formulation acts as a disinfectant for all types of hard surfaces as well as for fogging. In particular, the disinfectant formulation of the invention comprising DDAC and copper which exhibits both anti-microbial and anti-viral activity. The disinfectant with 130× and 225× dilution with water is able to kill 100% and 98% of bacterial strain E. coli (ATCC 8739) in 10 minutes respectively. The disinfectant with dilution more than 10× shows no erythema, no edema, no inflammation and skin allergy.

### FIELD OF INVENTION

The present invention relates to a disinfectant and its methods of preparation. In particular, the invention relates to unique disinfectant and its method of preparation comprising didecyldimethylammonium chloride with metal ion structures in ionic liquid. The disinfectant having anti-microbial and anti-viral properties for air and surface disinfection.

### BACKGROUND ART

The unregulated use of antibiotics is the foremost cause of increase in the development of new resistance mechanisms in drug resistant pathogens. Multidrug-resistant bacteria are commonly observed in healthcare associated infections and lead to high mortality. According to a study, approximately 20% of all reported pathogens show multidrug-resistant patterns. For instance, methicillin-resistant Staphylococcus aureus, vancomycin-resistant Enterococcus (VRE) and multi-drug resistant of Pseudomonas aeruginosa.

Generally, pathogens are transmitted through direct or indirect contact. It is observed that the microorganisms that are transmitted through contact mode are multidrug-resistant bacteria for example, MRSA, ESBL-producing Gram-negative organisms. C. difficile, and rotavirus. C. difficile is the most commonly reported pathogen in United States hospitals.

Droplet transmission may occur when microorganisms are transmitted from the respiratory tract by large droplets (greater than 5 microns) and travel less than 3 feet. Airborne transmission involves the transmission of organisms from the respiratory tract by small droplets (less than 5 microns) that travel long distances. Chickenpox virus, tuberculosis, measles, and the novel SARS-COV-2 virus may be transmitted through the airborne route.

Metal ions are known to inactivate viruses or bacteria by interacting with cells either directly or indirectly. The binding mechanism of metal ions to biological macromolecules is interpreted through site-specific Fenton mechanism. In the vicinity of their targets, secondary radicals are also formed. A large number of hydroxyl radicals are generated in repeated cyclic redox reactions and these generated radicals cause multi-hit damage to the targets. Cell death or viral dysfunction can also occur when protein structures are altered so they would not be able to perform their normal function.

Copper is chosen as the preferred metal ion because it has the ability to easily accept and donate electrons and because of this property, Cu (II) offers interesting possibilities in disinfection applications. Literature reports reveal that Cu (II) is effective for shifting of iron from iron-sulphur clusters, and plays an important role in protein binding sites. It is known to target the respiratory enzymes of E. coli in the cell membrane. Thus, the enzyme structure and function get altered due to binding of copper with the thiol group on proteins. The chelating of metal ions alters the molecular structure and further hinders decarboxylation of carboxylic acids. In comparison to conventional antibiotics, bacteria are considerably less likely to develop resistance to metal ions because metals can act on an extensive range of microbial targets, and to resist their antimicrobial activity, the microbe will need to develop many mutations.

Copper has been used in surface disinfection in the form of copper alloys which are capable of eliminating 99.9% morbific bacteria within two hours of contact at room temperature, which includes methicillin resistant Staphylococcus aureus, Pseudomonas aeruginosa, Campylobacter jejuni, Listeria monocytogenes, Legionella pneumophila, Escherichia coli, Mycobacterium tuberculosis, Salmonella enterica. These bacteria are observed to be the most hazardous pathogens capable of causing severe and repeatedly fatal infections.

In early 2021, the U.S. Environmental Protection Agency (EPA) reported that copper alloys in a sufficient amount can provide long-term effectiveness against viruses, including SARS-COV-2.

However, the copper based aqueous formulations suffers from less stability and which makes it difficult to scale up the copper-based formulation at industrial level.

Therefore, in order to overcome the above mention drawbacks, we are disclosing a novel disinfectant and its formulation comprising copper ions in combination with the other anti-microbial agent, that has improved stability and high effectiveness against the microbes present on any surface.

### DISCLOSURE OF THE INVENTION

The present invention discloses a disinfectant comprising
i. sodium chloride in the range of 0.01% to 0.05%;
ii. ethyl alcohol in the range of 0.50% to 2.0%;
iii. sodium dodecyl sulphate in the range of 0.13% to 0.17%;
iv. hydroxybenzaldehyde in the range of 0.10% to 0.14%;
v. ionic liquid in the range of 1.15% to 1.25%;
vi. copper salt in the range of 0.20% to 0.60%;
vii. didecyldimethylammononium chloride in the range of 4.80% to 5.02%; and
viii. water in the range of 91.0% to 94.0%.

The method of preparation of disinfectant comprises followings solution A: quaternary ammonium compound solution in a range of 2-12% in water.
solution B: the solution of copper salt in a solvent.
solution C: solution of sodium hydroxide and different amounts (5-75 g) of p-hydroxybenzaldehyde/m-hydroxybenzaldehyde/salicylaldehyde/or their combinations thereof.

Take 4.89% of solution A and 10-30 ml of solution C along with 1.5 g of any surfactant in water to make solution D. To solution D, add 5-20 ml of solution B to obtain a uniform solution. Add 5-23 g of ionic liquid/or their combinations. Dilute the contents with water to make 1 L volume of total solution.

### BRIEF DESCRIPTION OF DRAWINGS AND TABLES

The foregoing and other features and advantages of the invention will be more fully understood from the following descriptions made with reference to the figures.

FIG. 1 illustrates the tabulated result of the time-kill assay performed for HP08-04 where the disinfectant formulation has been diluted 100 times, HP08-08 where the formulation has been diluted 130 times and HP08-09 where the formulation has been diluted 225 times. HP08-04 was 100% effective at 30 seconds, HP08-08 was 100% effective at 10 minutes whereas HP08-09 was 98% effective at 10 minutes.

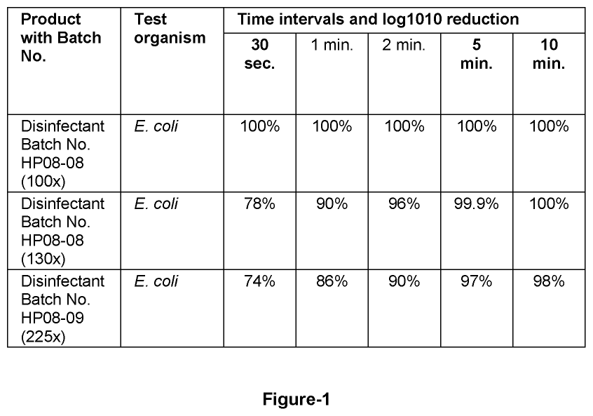

FIG. 2 illustrates the residual efficacy of HP08-01, HP08-02, HP08-03, HP08-05, HP08-06, HP08-07 and HP08-04.

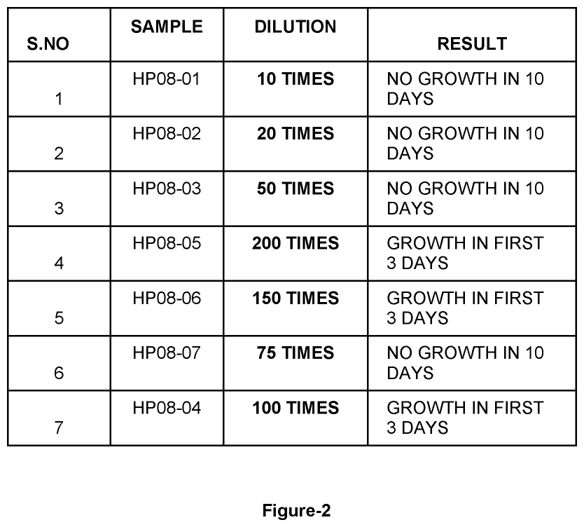

Table 1. Standard for skin irritation study

Table 2. Dermal irritation study of HP08 10× and 20× at different time intervals in rabbits.

These and other features and advantages of the present invention may be incorporated into certain embodiments of the invention and will become more fully apparent from the following description or may be learned by the practice of the invention as set forth hereinafter. The present invention does not require that all the advantageous features and all the advantages described herein be incorporated into every embodiment of the invention.

### BEST MODE(S) FOR CARRYING OUT THE INVENTION

The following presents a simplified description of the invention to provide a basic understanding of some aspects of the invention. This description is not an extensive overview of the present invention. It is not intended to identify the key/critical elements of the invention or to delineate the scope of the invention. Its sole purpose is to present some concept of the invention in a simplified form.

Accordingly, those of ordinary skill in the art will recognize that various changes and modifications of the embodiments described herein can be made without departing from the scope of the invention. In addition, descriptions of well-known functions and constructions are omitted for clarity and conciseness.

Features that are described and/or illustrated with respect to one embodiment may be used in the same way or in a similar way in one or more other embodiments and/or in combination with or instead of the features of the other embodiments.

The terms and words used in the following description and claims are not limited to the bibliographical meanings, but, are merely used by the inventor to enable a clear and consistent understanding of the invention. Accordingly, it should be apparent to those skilled in the art that the following description of exemplary embodiments of the present invention are provided for illustration purpose only and not for the purpose of limiting the invention as defined by the appended claims and their equivalents.

As used herein, the singular forms “a,” “an,” and “the” include plural referents unless the context clearly dictates otherwise.

As used herein, the terminology “substantially” it is intended that the recited characteristic, parameter, or value need not be achieved exactly, but that deviations or variations, including for example, tolerances, measurement error, measurement accuracy limitations and other factors known to those of skill in the art, may occur in amounts that do not preclude the effect the characteristic was intended to provide.

As used herein, the terminology “comprises/comprising” when used in this specification is taken to specify the presence of stated features, integers, steps or components but does not preclude the presence or addition of one or more other features, integers, steps, components or groups thereof.

As used herein, the terminology “HP08” refers to the disinfectant formulation.

As used herein, the terminology “MBPC” when used in this specification is taken to specify the Minimum Biofilm Prevention Concentration.

As used herein, the terminology “MBIC” when used in this specification is taken to specify the Minimum Biofilm Inhibition Concentration.

Reference will be made in detail to embodiments of the present disclosure. The same or similar elements and the elements having same or similar functions are denoted by reference numerals throughout the descriptions. The embodiments described herein with reference to drawings are explanatory, illustrative, and used to generally understand the present disclosure. The embodiments shall not be construed to limit the present disclosure.

The present invention provides a disinfectant having antimicrobial and antiviral properties.

According to embodiments of the present invention, it provides for anti-microbial and anti-viral formulation for surface disinfection.

The disinfectant comprising
ix. sodium chloride in the range of 0.01% to 0.05%;
X. ethyl alcohol in the range of 0.50% to 2.0%;
xi. sodium dodecyl sulphate in the range of 0.13% to 0.17%;
xii. hydroxybenzaldehyde in the range of 0.10% to 0.14%;
xiii. ionic liquid in the range of 1.15% to 1.25%;
xiv. copper salt in the range of 0.20% to 0.60%;
xv. didecyldimethylammononium chloride in the range of 4.80% to 5.02%; and
xvi. water in the range of 91.0% to 94.0%.

According to the embodiments of the present invention, the disinfectant formulation comprises quaternary ammonium compound and copper salts as active ingredients.

According to the embodiments of the present invention, the copper salt selected from the group consisting of copper chloride, copper sulphate, copper nitrate, copper acetate, copper perchlorate, copper iodide and/or their combinations thereof but not limited to these only.

In a preferred embodiment the copper salt is copper sulphate.

According to the embodiments of the present invention, the wherein ionic liquid is selected from the group consisting of Halides of 1-(carboxymethyl)pyridine-1-ium; 1,3-bis(carboxymethyl)-1H-benzo[d]imidazole-3-ium; 3-(carboxymethyl)-1-methyl-1H-imidazol-3-ium; 1,3-bis(carboxymethyl)-1H-imidazol-3-ium and/or their combinations thereof.

According to the embodiments of the present invention, the wherein hydroxybenzaldehyde is selected from the group consisting of p-hydroxybenzaldehyde, m-hydroxybenzaldehyde and salicylaldehyde or combinations thereof.

The quaternary ammonium compound according to embodiments of the present invention is DDAC.

According to embodiments, the disinfectant solution of the present invention comprises quaternary ammonium compound DDAC as one active entity.

In a preferred embodiment the disinfectant comprising
i. sodium chloride 0.02%;
ii. ethyl alcohol 1.03%;
iii. sodium dodecyl sulphate 0.162%;
iv. hydroxybenzaldehyde 0.113%;
v. ionic liquid 1.21%;
vi. copper salt 0.42%;
vii. didecyldimethylammononium chloride 4.89%; and
viii. water 93.15%

In a preferred embodiment the disinfectant comprising
ix. sodium chloride 0.02%;
x. ethyl alcohol 1.03%;
xi. sodium dodecyl sulphate 0.162%;
xii. hydroxybenzaldehyde 0.113%;
xiii. 1-(carboxymethyl)pyridine-1-ium; 1,3-bis(carboxymethyl)-1H-benzo[d]imidazole-3-ium 1.21%;
xiv copper sulphate 0.42%;
xv. didecyldimethylammononium chloride 4.89%; and
xvi. water 93.15%

In an embodiment the method of preparation of disinfectant comprises followings
solution A: quaternary ammonium compound solution in a range of 2-12% in water.
solution B: the solution of copper salt in a solvent.
solution C: solution of sodium hydroxide and different amounts (5-75 g) of p-hydroxybenzaldehyde/m-hydroxybenzaldehyde/salicylaldehyde/or their combinations thereof.

In an embodiment take 4.89% of solution A and 10-30 ml of solution C along with 1.5 g of any surfactant in water to make solution D. To solution D, add 5-20 ml of solution B to obtain a uniform solution. Add 5-23 g of ionic liquid/or their combinations. Dilute the contents with water to make 1 L volume of total solution.

In a preferred embodiment the method of preparation of disinfectant comprises followings
solution A: quaternary ammonium compound solution in a range of 4% in water.
solution B: the solution of copper salt 0.42% in a solvent.
solution C: solution of sodium hydroxide and 25 g of p-hydroxybenzaldehyde.

In an embodiment take the 40% of solution A and 20 ml of solution C along with 1.5 g of any surfactant in water to make solution D. To solution D, add 10 ml of solution B to obtain a uniform solution. Add 15 g of ionic liquid/or their combinations. Dilute the contents with water to make 1 L volume of total solution.

In an embodiment the preparation of disinfectant occurs in the presence of ionic liquid/s.

The ionic liquids according to embodiments of the present invention can be selected from the group comprising Halides of 1-(carboxymethyl)pyridine-1-ium; 1,3-bis(carboxymethyl)-1H-benzo[d]imidazole-3-ium; 3-(carboxymethyl)-1-methyl-1H-imidazol-3-ium; 1,3-bis(carboxymethyl)-1H-imidazol-3-ium and/or their combinations thereof.

The prepared disinfectant has been fully characterized and confirmed by UV-Vis Spectroscopy and Fourier-transform infrared spectroscopy (FT-IR) techniques.

The disinfectant formulation or solution of the present invention is focused on developing a material that prevents the spread of microbial infections. The efficacy of the invention was determined by means of the following examples:

### Example 1: Efficacy Study: Time-Kill Assay

The time-kill kinetics assay is used to evaluate the bactericidal or bacteriostatic activity of an antimicrobial agent against a bacterial strain over time. Bactericidal activity is defined as >3 log 10 10-fold decrease in colony forming units (surviving bacteria), which is equivalent to 99.9% killing of the inoculum.

Antimicrobial effectiveness of three dilutions of the disinfectant with Batch No. HP08-04 (100×), HP08-08 (130×) and HP08-09 (225×) were determined against bacterial strain E. coli (ATCC 8739) at different time intervals (30 seconds, 1 minute, 2 minutes, 5 minutes and 10 minutes) by time-kill assay. Here, the disinfectant formulation (HP08) has been diluted 100 times, 130 times and 225 times respectively. 1 mL of the sample was challenged with 105 CFU/mL of the cell suspension of the target organism. For this, a cell suspension was prepared from 18 to 24 hours old culture of E. coli in 9 mL 0.9% saline. Serial dilutions were prepared to get the known cell count and 0.1 mL of the dilution containing not more than 107 cells was added into the tube containing 1 mL of the product. Post-addition of culture to the products, contact time intervals of 30 seconds, 1 minute, 2 minutes, 5 minutes and 10 minutes were maintained. After each time interval, the entire 1 mL of the product was transferred into 9 mL of Dey Engley neutralizing broth. Serial dilutions were prepared and plating was done by using SCDA media. Plates were incubated at 32.5° C. for 18 to 24 hours. After incubation, the plates were observed for recovery of the test organism. Positive control was also maintained along with the sample. The recovery of the test organism at different intervals was compared with positive control and log 10 reduction was calculated.

Complete killing or greater than 6 log 10 reduction (>99.9999%) was observed with disinfectant Batch no. HP08-04 (100×) at 30 seconds time interval.

3 log 10 reduction (>99.9%) was observed with disinfectant Batch no. HP08-08 (130×) at 10 minutes time interval followed by 3 log 10 reduction (99.9%) at 5 minutes and 2 log 10 reduction (90%) at 1 minute time interval.

Batch No. HP08-09 (225×) showed maximum log 10 reduction i.e., >3 log 10 reductions (<99%) at 10 minutes time interval.

### Example 2: Residual Efficacy

Protocol to test residual efficacy of HP08 was used as per specifications provided under USP-1072 Disinfectants and Antiseptics and ISO 14698 (1-3). The efficacy of HPO8 against Escherichia coli, Staphylococcus aureus and Candida albicans was checked at various dilutions. It was found that HPO8 diluted 75 times demonstrated no growth up to 10 days. Microbial growth limit for the test was NLT 95.0%.

### Evaluation of Antibiofilm Activity

The antibiofilm activity of the test substance(s) against S. aureus ATCC 29213 biofilms was determined in 96 well flat-bottomed microtiter plates by XTT dye reduction test. For prevention studies, 200 μl of the microbial suspension (5. a11re11s ″″107 cfu/ml) was incubated for biofilm formation in the nutrient medium [S. aureus, Mueller Hinton Broth (MHB)] containing two-fold serial dilutions of the test substance (HP08-02, 1:2 to 1:4096 dilution) for 24 h at 37° C.

Untreated controls were set up in parallel. After incubation, the biofilms formed were washed twice with normal saline and exposed to XTT-menadione solution (XTT, 0.5 mg/ml; menadione, 10 μM) for 1 h at 37° C. in dark. The absorbance of the supernatants was measured at 450 nm. Likewise, to determine the efficacy of the test substance in eradicating the pre-existing biofilms, 24 h old biofilms of S. aureus ATCC 29213 was treated with two-fold serial dilutions of the lest substance for 24 h at 37° C., and quantified by XTT dye reduction test. MBPC₅₀, MBPC₉₀ and MBPC₁₀₀ were defined as the concentrations of the test substance that led to 50%, 90% and 100% decrease in biofilm formation respectively relative to the untreated controls. MBPC₅₀, MBPC₉₀ and MBPC₁₀₀ were defined as the concentrations of test substance that led to 50, 90% and 100% reduction in pre-formed biofilm biomass respectively compared with the untreated controls.

MBPC₅₀, MBPC₉₀ and MBPC₁₀₀ at dilution of 1:512; 1:256; 1:256.

The present invention relates to disinfectant for inhibiting or removing micro-organisms from surfaces.

### Skin Irritation HP08 as Strength 10×, 20×.

For observing the skin compatibility of the invented product, the skin irritation assay has been performed according to the OECD guidelines. For this the acute singled dermal irritation study was performed, control group of rabbits received placebo patch and a treated group of rabbits received HP08 loaded transdermal patch. For experimental use approximately 5 cm×5 cm of rabbit's trunk was unclipped. After that the invented product was applied under a 2.5 cm×2.5 cm gauze patch was applied on one integral site per rabbit and wrapped with an occlusive dressing. The tested product was attached to skin for 4 h and after that remove all the patches and wrappings. Thereafter, wash the test site with lukewarm water to remove the remaining tested product on the skin. No dermal reactions were observed for 4 h after removing the patch.

Subsequently, according to the guidelines, the erythema and edema were scored on a scale of 0-4, where 0 shows no effect and 4 number represents severe symptoms. After removal of the patches each animal, dermal response were scores at time period of 1 h, 24 h, 48h and 72 h. The score was divided by three to obtain a mean irritation score per time point. The results of both groups (placebo and tested) were compared. The table-1 provided the mean scores and averaged to obtain the primary irritation index.

TABLE 1
Standard for skin irritation study

| Skin responses | Score |
|---|---|
| Erythema and Eschar formation | |
| No erythema | 0 |
| Very slight erythema (barely perceptible) | 1 |
| Well defined | 2 |
| Moderate to serve erythema | 3 |
| Severe erythema to slight eschar formation | 4 |
| Total Possible erythema score | 4 |
| Edema formation | |
| No edema | 0 |
| Very slight edema (barely perceptible) | 1 |
| Slight edema | 2 |
| Moderate edema | 3 |
| Severe edema | 4 |
| Total possible edema score | 4 |

TABLE 2
Dermal irritation study of HP08 10× and
20× at different time intervals in rabbits.

Score

Acute singled dermal irritation
study 10× 20×

| | | | |
|---|---|---|---|
| Erythema | 0 | 0 | 0 |
| 1 h after removal of patches | 0 | 0 | 0 |
| 24 h after removal of patches | 0 | 0 | 0 |
| 48 h after removal of patches | 0 | 0 | 0 |
| 72 h after removal of patches | 0 | 0 | 0 |
| Edema | 0 | 0 | 0 |
| 1 h after removal of patches | 0 | 0 | 0 |
| 24 h after removal of patches | 0 | 0 | 0 |
| 48 h after removal of patches | 0 | 0 | 0 |
| 72 h after removal of patches | 0 | 0 | 0 |

### Skin Irritation Classification

Non-irritant—Compound producing combined averages (primary irritation indices) of 0.
Mild irritation—Compound producing combined averages of less than 2
Moderate irritation—Compounds producing combined averages of 2 to 5
Severe or primary irritant—Compound producing combined averages greater than 5 or with extensive eschar information.

No erythema, no edema, no inflammation, and skin allergy found. The compounds are therefore classified as non-irritants.

Although the embodiments herein are described with various specific embodiments, it will be obvious for a person skilled in the art to practice the invention with modifications. However, all such modifications are deemed to be within the scope of the invention.

### INDUSTRIAL APPLICABILITY

the invention relates to unique disinfectant and its method of preparation comprising didecyldimethylammonium chloride with metal ion structures in ionic liquid.

The present invention provides a disinfectant having antimicrobial and antiviral properties.

The formulation method can be scaled up at industrial scale.

The disinfectant has improved stability and high effectiveness against the microbes present on any surface.

100× and 130× dilution (with water) of disinfectant is able to kill 100% of bacterial strain E. coli (ATCC 8739) in 30 seconds and 10 minutes respectively.

75× dilution disinfectant shows no growth of Escherichia coli, Staphylococcus aureus or/and Candida albicans in a solution up to 10 days.

10× shows no erythema, no edema, no inflammation, and skin allergy.

### REFERENCES

i. Poonam Singla, Priyanka Dalal, Mahaldeep Kaur, Geeta Arya, Surendra Nimesh, Rachna Singh, Deepak B. Salunke. Bile Acid Oligomers and Their Combination with Antibiotics To Combat Bacterial Infections, Journal of Medicinal Chemistry, 2018
ii. R. L. Morgan. Skin Irritation Testing in Rabbits Complicated by Dermal Mucormycosis, Toxicologic Pathology, 1985
iii. Ronald L. Morgan, Thomas R. Castles, Gary M. Zwicker, Dee O. N. Taylor. Skin Irritation Testing in Rabbits Complicated by Dermal Mucormycosis, Toxicologic Pathology, 2016
iv. S. Stohs, D. Bagchi. Oxidative mechanisms in the toxicity of metal ions. Free Radical Biology and Medicine, 1995, 18, 321
v. G. McDonnell, A. D. Russell. Antiseptics and Disinfectants: Activity, Action, and Resistance. Clinical Microbiology Reviews, 1999, 12, 147
vi. R. W. Hay. Metal ion-catalyzed decarboxylations of biological interest, in Metal lons in Biological Systems, Vol. 5, Sigel, H., Ed., Marcel Dekker, New York, 1976, 127
vii. R. B. Martin. Acid-catalyzed ester hydrolysis, J. Am. Chem. Soc., 89, 2501, 1967
viii. J. W. Huff, K. S. Sastry, M. P. Gordon, and W. E. C. Wacker. The action of metal ions on tobacco mosaic virus ribonucleic acid, Biochemistry, 3, 501, 1964
ix. G. Grass, C. Rensing, and M. Solioz, Metallic copper as an antimicrobial surface. Applied and Environmental Microbiology, 2011, 77, 1541
x. G. Bitton, and V. Friehofer. Influence of extracellular polysaccharides on the toxicity of copper and cadmium toward Klebsiella aerogenes, Microbial Ecology, 4, 119, 1978
xi. (a) D. A. Cooksey. Characterization of a copper resistance plasmid conserved in copper-resistant strains of Pseudomonas syringae pv. tomato, Applied and Environmental Microbiology, 53, 454, 1987. (b) A. Totsuka, and K. Ohtaki. The effects of amino acids and metals on the infectivity of poliovirusribonucleic acid, Japanese Journal of Microbiology, 18, 107, 1974
xii. H. Babich, and G. Stotzky. Environmental factors that influence the toxicity of heavy metal and gaseous pollutants to microorganisms, CRC Critical Reviews in Microbiology, 8, 99, 1980
xiii. A. Singh, M. W. LeChevallier, and G. A. McFeters. Reduced virulence of Yersinia enterocolitica by copper-induced injury, Applied and Environmental Microbiology, 50, 406, 1985.
xiv. B. Bagchi, S. Kar, S. Dey, S. Bhandary, D. Roy, and T. Mukhopadhyay. In situ synthesis and antibacterial activity of copper nanoparticle loaded natural montmorillonite clay based on contact inhibition and ion release. Colloids Surf. B. Biointerfaces., 2013, 108, 358. (b) S. L. Warnes, C. J. Highmore, and C. W. Keevil. Horizontal transfer of antibiotic resistance genes on abiotic touchsurfaces: implications for public health. mBio, 2013, 3, e00489
xv. C. E. Santo, E. W. Lam, and C. G. Elowsky. Bacterial killing by dry metallic copper surfaces. Applied and Environmental Microbiology, 2011, 77, 794
xvi. Morones, J. Elechiguerra, and A. Camacho. The bactericidal effect of silver nanoparticles. Nanotechnology, 2005, 16, 2346
xvii. A. E. Nel, L. Madler, and D. Velegol. Understanding biophysicochemical interactions at the nano-bio interface. Nature Materials, 2009, 8, 543
xviii. R. Allaker, and K. Memarzadeh. Nanoparticles and the control of oral infections. International Journal of Antimicrobial Agents., 2014, 43, 95

We claim:

1. A disinfectant comprising
i. sodium chloride in the range of 0.01% to 0.05%;
ii. ethyl alcohol in the range of 0.50% to 2.0%;
iii. sodium dodecyl sulphate in the range of 0.13% to 0.17%;
iv. hydroxybenzaldehyde in the range of 0.10% to 0.14%;
v. ionic liquid in the range of 1.15% to 1.25%;
vi. copper salt in the range of 0.20% to 0.60%;
vii. didecyldimethylammononium chloride in the range of 4.80% to 5.02%; and
viii. water in the range of 91.0% to 94.0%.

2. The disinfectant as in claim 1, wherein copper salt selected from the group consisting of copper chloride, copper sulphate, copper nitrate, copper acetate, copper perchlorate, copper iodide and/or their combinations thereof but not limited to these only.

3. The disinfectant as in claim 1, wherein ionic liquid is selected from the group consisting of Halides of 1-(carboxymethyl)pyridine-1-ium; 1,3-bis(carboxymethyl)-1H-benzo[d]imidazole-3-ium; 3-(carboxymethyl)-1-methyl-1H-imidazol-3-ium; 1,3-bis(carboxymethyl)-1H-imidazol-3-ium and/or their combinations thereof.

4. The disinfectant as in claim 1, wherein hydroxybenzaldehyde is selected from the group consisting of p-hydroxybenzaldehyde, m-hydroxybenzaldehyde and salicylaldehyde or combinations thereof.

5. The disinfectant as claimed in claim 1, prepare by a method comprising;
i. mix the 30-60% of solution A, 10-30 ml of solution C with 1.5 g of surfactant in water to make solution D;
ii. to solution D, add 5-20 ml of solution B to obtain a uniform solution;
iii. add 5-23 g of ionic liquid;
iv. dilute the contents with water to make 100% volume of total solution;
Wherein;
solution A: solution of quaternary ammonium compound in water;
solution B: solution of copper salt in ethanol and water;
solution C: solution of sodium hydroxide and hydroxybenzaldehyde.

6. The disinfectant as claimed in claim 1, with 100× and 130× dilution (with water) is able to kill 100% of bacterial strain E. coli (ATCC 8739) in 30 seconds and 10 minutes respectively.

7. The disinfectant as claimed in claim 1, with 75 × dilution shows no growth of Escherichia coli, Staphylococcus aureus or/and Candida albicans in a solution up to 10 days.

8. The disinfectant as claimed in claim 1, shows MBPC₅₀, MBPC₉₀ and MBPC₁₀₀ at dilution of 1:512; 1:256; 1:256 respectively in antibiofilm activity test.

9. The disinfectant as claimed in claim 1 with dilution of 10× shows no erythema, no edema, no inflammation and skin allergy.
